(19) United States
(12) Patent Application Publication
(10) Pub. No.: US 2001/0006633 A1
(43) Pub. Date: Jul. 5, 2001

(54) ADENOVIRUS-CHEMOTHERAPEUTIC COMBINATION FOR TREATING CANCER

(76) Inventor: DAVID KIRN, MILL VALLEY, CA (US)

Correspondence Address: GREGORY GIOTTA PHD, VICE PRESIDENT AND CHIEF LEGAL COUNSEL, ONYX PHARMACEUTICALS INC, 3031 RESEARCH DRIVE, RICHMOND, CA, 94806

(21) Appl. No.: 09/294,263

(22) Filed: Apr. 19, 1999

Related U.S. Application Data
(60) Provisional application No. 60/085,762, filed on May 15, 1998.

(51) Int. Cl. A61K 48/00; A61K 31/70

(52) U.S. Cl. 424/93.6; 435/320.1; 514/44

(57) ABSTRACT

Compositions and methods are described for treating or preventing cancer consisting of a combination of adenovirus and chemotherapy such that the combination acts synergesticaly to kill or prevent the growth of a cancer where the cancer is preferably recurrent or metastatic squamous cell cancer.

### FIELD OF THE INVENTION

The invention described herein relates generally to cancer, and to methods and compositions for treating or preventing cancer using adenovirus in combination with chemotherapy.

### BACKGROUND OF THE INVENTION

Viral therapy for treating cancer using replicating viruses has been tried over the years, unfortunately, though with little success. The most notable studies were clinical trials carried out during the 1960s and 1970s. One such study was the work of Southam and Moore. See, Southam, C. M., and Moore, A. E. Cancer 1952, vol. 5, pp. 1025-1034. In this study the authors used a strain of West Nile virus, Egypt 101, to treat patients having a number of different types of cancer. Unfortunately, the most promising study in lymphoma patients revealed that less than 10% of the patients exhibited neoplasm regression. Moreover, the virus caused viraemia in 90% of the patients, and a significant number also experienced encephalitis, a not surprising effect considering the neurotropic nature of the virus.

The second study was conducted by Smith and Collins in 1956. See, Smith, R. et al. Cancer (1956), vol. 9, pp. 1211-1218. They tested wild type adenovirus in 30 patients who had advanced epidermoid carcinoma of the cervix. Different amounts of virus were given by direct intraneoplasmal inoculation or by arterial perfusion. Different adenovirus serial types were used, and the patients pre-existing status of anti-adenovirus neutralizing antibody was determined prior to injection with the virus. It was observed that approximately 26 of the 40 viral inoculations resulted in an area of necrosis in the central portion of the injected pelvic neoplasm. It was also noted that there was no damage to normal pelvic tissue. Neoplasm necrosis occurred for a period of up to 30 days; however, in no case was the neoplasm totally destroyed. It was noted that patients with pre-existing anti-adenovirus antibody faired less well than patients with no immunity to the virus. This indicated that spread of the virus may be restricted by host immune response.

The third antiviral approach to treating cancer was conducted by Asada. See, Asada, Cancer (1974), vol. 34, pp 1907-1928. In this study mumps virus was used to treat patients with advanced cancer. The virus was administered to 90 patients with different malignancies. Little or no side effects were observed, and in 37 of the 90 patients, the neoplasm disappeared or regressed to less than half of its initial size. Additionally, minor responses were observed in 42 other patients. The virus seemed to act in two phases: in the first, which occurred a few days after injection, viral replication caused significant neoplasm destruction, and in the second, there was a subsequent period during which neoplasm re-growth was static. Unfortunately, though, as was observed in the other trials, in all cases the cancer eventually re-grew.

Considering the limited effectiveness of viral therapy displayed in these trials, it is not surprising that this approach was essentially dropped, with two notable recent exceptions. The first is the work of Martuza et al., relating to the use of herpes simplex for treating cancer (See, PCT/US96/08621). Here the strategy is to use replication-competent herpes simplex that expresses neoplasm or cell-specific transcriptional regulatory sequences which are operatively linked to an essential herpes simplex virus gene. Additionally, attempts have been made to render the virus non-neurovirulent through an appropriate mutation. Unfortunately, such mutants retain significant residual neurovirulence, and how useful they will ultimately be in treating cancer is uncertain.

The second approach is described in U.S. Pat. No. 5,677,178, inventor McCormick. This approach takes advantage of the loss of tumor suppressor proteins in cancer cells. Perhaps the most notable such tumor suppressor protein is p53. A function of p53 is to inhibit the progression of mammalian cells through the cell cycle in response to DNA damage. The e1b p55 protein of wild-type adenovirus binds to p53 in adenovirus infected cells that exhibit p53 and produce a substantial inactivation of p53 function. Functional adenoviral e1bp55 protein is essential for efficient adenoviral replication in cells containing functional p53. Adenovirus mutants which substantially lack the ability to bind p53 are replication deficient in non-replicating, non-neoplastic cells having normal levels of functional p53. However, such adenoviral mutants exhibit a replication phenotype in cells which are deficient in p53 function (for example, cells which are homozygous for substantially deleted p53 alleles, cells which comprise mutant p53 proteins which are essentially non-functional) and thus cause the death of such cells. In clinical trials that are still ongoing, an adenovirus mutant described above has been shown to be biologically active and cause partial tumor necrosis in head and neck cancer.

The viral therapy clinical trials of the 60s and 70s, although not an overt success, nevertheless set the foundation for the more recent work in this area. It is possible that the viral anticancer effects observed in these studies could be enhanced if viral treatment is combined with standard modalities for treating cancer, such as chemotherapy.

### SUMMARY OF THE INVENTION

A first object of the invention is to describe a method for treating cancer consisting of administering to a patient in need of such treatment a replicating adenoviral vector in combination with chemotherapy.

A second object of the invention is to describe a method for treating cancer consisting of administering to a patient in need of such treatment a replicating adenoviral vector in combination with chemotherapy wherein the combination causes an anti-cancer synergistic effect.

A third object of the invention is to describe a method for treating squamous cell cancer consisting of direct injection of adenovirus into the cancer and administration of a chemotherapeutic to produce a synergistic effect against the cancer.

A fourth object of the invention is to describe a method for treating squamous cell cancer consisting of direct injection of adenovirus into the cancer and administration of two chemotherapeutics, cisplatin and 5-fluorouracil, to produce a synergistic effect against the cancer.

A fifth object of the invention is to describe a method for treating squamous cell cancer of the head and neck consisting of direct injection of adenovirus into the cancer and administration of two chemotherapeutics cisplatin and 5-fluorouracil, to produce a synergistic effect against the cancer.

A sixth object of the invention is to describe compositions consisting of adenovirus and chemotherapeutics that exert a synergistic effect against cancer.

A seventh object of the invention is to describe compositions consisting of adenovirus and two chemotherapeutics, cisplatin and 5-fluorouracil, that exert a synergistic effect against cancer.

These and other objects of the present invention will become apparent to one of ordinary skill in the art upon reading the description of the various aspects of the invention in the following specification. The foregoing and other aspects of the present invention are explained in greater detail in the drawings, detailed description, and examples set forth below.

### DETAILED DESCRIPTION OF THE INVENTION

All publications and patent applications mentioned in this specification are herein incorporated by reference to the same extent as if each individual publication or patent application was specifically and individually indicated to be incorporated by reference.

Definitions

Unless defined otherwise, all technical and scientific terms used herein have the same meaning as commonly understood by one of ordinary skill in the art to which this invention belongs. Generally, the nomenclature used herein and the laboratory procedures described below are those well known and commonly employed in the art. Standard techniques are used for recombinant nucleic acid methods, polynucleotide synthesis, and microbial culture and transformation (e.g., electroporation, lipofection). Generally enzymatic reactions and purification steps are performed according to the manufacturer's specifications. The techniques and procedures are generally performed according to conventional methods in the art and various general references (see generally, Sambrook et al., Molecular Cloning: A Laboratory Manual, 2nd. edition (1989) Cold Spring Harbor Laboratory Press, Cold Spring Harbor, N.Y., which is incorporated herein by reference) which are provided throughout this document. The nomenclature used herein and the laboratory procedures in analytical chemistry, organic synthetic chemistry, and pharmaceutical formulation described below are those well known and commonly employed in the art. Standard techniques are used for chemical syntheses, chemical analyses, pharmaceutical formulation and delivery, and treatment of patients.

As employed throughout the disclosure, the following terms, unless otherwise indicated, shall be understood to have the following meanings:

The term “adenovirus” indicates over 40 adenoviral subtypes isolated from humans, and as many from other mammals and birds. See, Strauss, “Adenovirus infections in humans,” in The Adenoviruses, Ginsberg, ed., Plenum Press, New York, N.Y., pp. 451-596 (1984). The term preferably applies to two human serotypes, Ad2 and Ad5.

“Neoplastic cells” or “neoplasia” refer to cells which exhibit relatively autonomous growth, so that they exhibit an aberrant growth phenotype characterized by a significant loss of control of cell proliferation. Neoplastic cells comprise cells which may be actively replicating or in a temporary non-replicative resting state (G₁ or G₀); similarly, neoplastic cells may comprise cells which have a well-differentiated phenotype, a poorly-differentiated phenotype, or a mixture of both type of cells. Thus, not all neoplastic cells are necessarily replicating cells at a given timepoint. The set defined as neoplastic cells consists of cells in benign neoplasms and cells in malignant (or frank) neoplasms. Herein frankly neoplastic cells are frequently referred to as cancer, or cancer cells, typically termed carcinoma if originating from cells of endodermal or ectodermal histological origin, or sarcoma if originating from cell types derived from mesoderm.

“Physiological conditions,” or “physiological solution” refers to an aqueous environment having an ionic strength, pH, and temperature substantially similar to conditions in an intact mammalian cell or in a tissue space or organ of a living mammal. Typically, physiological conditions comprise an aqueous solution having about 150 mM NaCl, pH 6.5-7.6, and a temperature of approximately 22-37° C. Generally, physiological conditions are suitable binding conditions for intermolecular association of biological macromolecules. For example, physiological conditions of 150 mM NaCl, pH 7.4, at 37° C. are generally suitable.

Chemistry terms herein are used according to conventional usage in the art, as exemplified by The McGraw-Hill Dictionary of Chemical Terms (ed. Parker, S., 1985), McGraw-Hill, San Francisco, incorporated herein by reference.

DNA regions are operably linked when they are functionally related to each other. For example: a promoter is operably linked to a coding sequence if it controls the transcription of the sequence; a ribosome binding site is operably linked to a coding sequence if it is positioned so as to permit translation. Generally, operably linked means contiguous and, in the case of leader sequences, contiguous and in reading frame.

By replicating adenoviral vector is meant adenovirus or a mutant thereof that is capable of replicating in cancer cells. Such may include wild-type adenovirus, or, as discussed more in detail below, mutants of adenovirus that are capable of selecting replicating in certain types of cancer cells, preferrably those that lack one or more cancer suppressor proteins.

Without intending to be bound to a particular theory that would explain the synergistic activity of adenovirus and chemotherapy, it is suggested that the preferred combination of adenovirus and chemotherapy is an adenovirus E1b-mutant, in combination with cisplatin and 5-fluorouracil (5-Fu).

Adenovirus

It is noteworthy that while the instant invention is described in terms of adenovirus type 5, it may be practiced with other similar adenovirus serotypes. The general organization of the adenoviral genome is conserved among serotypes, and specific functions are similarly situated. Further, the adenovirus 5 genome is registered as Genbank accession #M73260, and the virus is available from the American Type Culture Collection, Rockville, Md., U.S.A., under accession number VR-5. Methods for the construction of adenoviral mutants are generally known in the art. See, Mittal, S. K., Virus Res., 1993, vol: 28, pages 67-90. Certain of the materials and methods used to construct adenovirus mutants are described by Hanke, T., et. al. (1990) Virology, vol. 177, pages 437-444, and Bett, A. J., et. al., (993) J. Virol. vol. 67, pages 5911-5921, and in PCT/CA96/00375. Microbix Biosystems, Inc., located at 341 Bering Avenue, Toronto, Ontario Canada, sells many of the materials used to construct adenovirus mutants, and provides Product Information Sheets on how to make them.

A preferred adenovirus mutant that can be used in combination with chemotherapy to produce the anti-cancer synergistic effect noted herein is one that lacks the capacity to express a viral protein that inactivates p53. Such proteins are encoded at least by the E1B and E40RF6 regions of the adenoviral genome. A function of the cellular phosphoprotein p53 is to inhibit the progression of mammalian cells through the cell cycle. Wild-type adenovirus E1b p55 protein binds to p53 in infected cells that have p53 and produce a substantial inactivation of p53 function, likely by sequestering p53 in an inactive form. Functional E1b p55 protein is essential for efficient adenoviral replication in cells containing functional p53. Hence, adenovirus variants which substantially lack the ability to bind p53 are replication deficient in non-replicating, non-neoplastic cells having normal levels of functional p53.

Human cancer cells frequently are homozygous or heterozygous for mutated (e.g., substitution, deletion, frameshift mutants) p53 alleles, and lack p53 function necessary for normal control of the cell cycle (Hollstein et al. (1991) Science 253: 49; Levine et al. (1991) op. cit., incorporated herein by reference). Thus, many neoplastic cells are p53⁽⁻⁾, either because they lack sufficient levels of p53 protein and/or because they express mutant forms of p53 which are incapable of substantial p53 function, and which may substantially diminish p53 function even when wild-type p53 may be present (e.g., by inhibiting formation of functional multimers). Some neoplastic cells may comprise alleles encoding essentially wild-type p53 proteins, but may comprise a second site mutation that substantially abrogates p53 function, such as a mutation that results in p53 protein being localized in the cytoplasm rather than in the nucleus; such second site mutants also substantially lack p53 function.

It is believed that replication deficient adenovirus species which lack the capacity to complex p53 but substantially retain other essential viral replicative functions will exhibit a replication phenotype in cells which are deficient in p53 function (e.g., cells which are homozygous for substantially deleted p53 alleles, cells which comprise mutant p53 proteins which are essentially nonfunctional) but will not substantially exhibit a replicative phenotype in non-replicating, non-neoplastic cells. Such replication deficient adenovirus species are referred to herein for convenience as E1b-p53⁽⁻⁾ replication deficient adenoviruses.

A cell population (such as a mixed cell culture or a human cancer patient) which comprises a subpopulation of neoplastic cells lacking p53 function and a subpopulation of non-neoplastic cells which express essentially normal p53 function can be contacted under infective conditions (i.e., conditions suitable for adenoviral infection of the cell population, typically physiological conditions) with a composition comprising an infectious dosage of a E1b-p53⁽⁻⁾ replication deficient adenovirus. Such contacting results in infection of the cell population with the E1b-p53⁽⁻⁾ replication deficient adenovirus. The infection produces preferential expression of a replication phenotype in a significant fraction of the cells comprising the subpopulation of neoplastic cells lacking p53 function but does not produce a substantial expression of a replicative phenotype in the subpopulation of non-neoplastic cells having essentially normal p53 function. The expression of a replication phenotype in an infected p53⁽⁻⁾ cell results in the death of the cell, such as by cytopathic effect (CPE), cell lysis, apoptosis, and the like, resulting in a selective ablation of neoplastic p53⁽⁻⁾ cells from the cell population.

Typically, E1b-p53⁽⁻⁾ replication deficient adenovirus constructs suitable for selective killing of p53(−) neoplastic cells comprise mutations (e.g., deletions, substitutions, frameshifts) which inactivate the ability of the E1b p55 polypeptide to bind p53 protein effectively. Such inactivating mutations typically occur in the regions of p55 which bind p53. Optionally, the mutant E1b region may encode and express a functional p19 protein encoded by the E1b region remains and that is functional in transactivation of adenoviral early genes in the absence of E1a polypeptides.

Suitable E1b-p53⁽⁻⁾ replication deficient adenovirus constructs for use in the methods and compositions of the invention include, but are not limited to the following examples: (1) adenovirus type 2 d1 1520, which contains a C to T mutation at nucleotide position 2022 that generates a stop codon 3 amino acids downstream of the AUG codon used for initiation of translation of the p55 protein and a deletion between nucleotides 2496 and 3323 replaced with a small linker insertion that generates a second stop codon at nucleotide 3336; the expression of the p19 protein is essentially unaffected (Barker and Berk (1987) Virology 156: 107, incorporated herein by reference, and (2) a composite adenovirus construct comprising adenovirus type 2 d1 1520 comprising at least the position 2022 mutation and/or the 2496-3323 deletion mutation, or a substantial portion thereof, and an additional mutation in p19 to yield a p19 cyt mutant; the composite virus construct lacks p55 and comprises the enhanced cytopathic effect of the p19 cyt mutation. A d2 d1 1520 is available from Dr. A. Berk, University of California at Los Angeles, Los Angeles, Calif., and is described in the literature, including Barker and Berk (1987) Virology 156:107.

It may be preferable to incorporate additional mutations into such adenovirus constructs to inhibit formation of infectious virions in neoplastic cells which otherwise would support replication of the E1b-p53⁽⁻⁾ mutants. Such additional inactivating mutations would be preferred in therapeutic modalities wherein complete viral replication forming infectious virions capable of spreading to and infecting adjacent cells is undesirable. These fully inactivated mutants are referred to as nonreplicable E1b-p53⁽⁻⁾ mutants. Such nonreplicable mutants comprise mutations which prevent formation of infectious virions even in p53⁽⁻⁾RB⁽⁻⁾ cells; such mutations typically are structural mutations in an essential virion protein or protease.

However, in many modalities it is desirable for the mutant virus to be replicable and to form infectious virions containing the mutant viral genome which may spread and infect other cells, thus amplifying the antineoplastic action of an initial dosage of mutant virus.

Additional E1b⁽⁻⁾ mutants lacking the capacity to bind p53 can be generated by those of skill in the art by generating mutations in the E1b gene region encoding the p55 polypeptide, expressing mutant p55 polypeptides, contacting the mutant p55 polypeptides with p53 or a binding fragment of p53 under aqueous binding conditions, and identifying mutant E1b polypeptides which do not specifically bind p53 as being candidate E1b⁽⁻⁾ mutants suitable for use in the invention.

It is noteworthy, that regardless of the desired adenovirus, wild type or a mutant thereof, associated cell selective cancer killing may be enhanced by targeting the virus to the cancer cells by altering its external binding proteins using the methods described in PCT/US96/01957, or by constructing into the virus tissue specific promoters (see, PCT/US95/14461) that drive the expression of certain pro-drug activator genes (see, PCT/GB95/00322).

Formulations

Adenovirus, including adenoviral mutants, may be formulated for therapeutic and diagnostic administration to a patient. For therapeutic or prophylactic uses, a sterile composition containing a pharmacologically effective dosage of adenovirus is administered to a human patient or veterinary non-human patient for treatment, for example, of a neoplastic condition. Generally, the composition will comprise about 10³ to 10¹⁵ or more adenovirus particles in an aqueous suspension. A pharmaceutically acceptable carrier or excipient is often employed in such sterile compositions. A variety of aqueous solutions can be used, eg. water, buffered water, 0.4% saline, 0.3% glycine and the like. These solutions are sterile and generally free of particulate matter other than the desired adenoviral vector. The compositions may contain pharmaceutically acceptable auxiliary substances as required to approximate physiological conditions such as pH adjusting and buffering agents, toxicity adjusting agents and the like, for example sodium acetate, sodium chloride, potassium chloride, calcium chloride, sodium lactate, etc. Excipients which enhance infection of cells by adenovirus may be included.

Adenoviruses of the invention, or the DNA contained therein, may also be delivered to neoplastic cells by liposome or immunoliposome delivery; such delivery may be selectively targeted to neoplastic cells on the basis of a cell surface property present on the neoplastic cell population (e.g., the presence of a cell surface protein which binds an immunoglobulin in an immunoliposome). Typically, an aqueous suspension containing the virions are encapsulated in liposomes or immunoliposomes. For example, a suspension of adenovirus virions can be encapsulated in micelles to form immunoliposomes by conventional methods (U.S. Pat. Nos. 5,043,164, 4,957,735, 4,925,661; Connor and Huang (1985) J. Cell Biol. 101: 582; Lasic DD (1992) Nature 355: 279; Novel Drug Delivery (eds. Prescott L F and Nimmo W S: Wiley, New York, 1989); Reddy et al. (1992) J. Immunol. 148: page 1585). Immunoliposomes comprising an antibody that binds specifically to a cancer cell antigen (e.g., CALLA, CEA) present on the cancer cells of the individual may be used to target virions, or virion DNA to those cells.

The compositions containing the present adenoviruses or cocktails thereof can be administered for prophylactic and/or therapeutic treatments of neoplastic disease. In therapeutic application, compositions are administered to a patient already affected by the particular neoplastic disease, in an amount sufficient to cure or at least partially arrest the condition and its complications. An amount adequate to accomplish this is defined as a “therapeutically effective dose” or “efficacious dose.” Amounts effective for this use will depend upon the severity of the condition, the general state of the patient, and the route of administration.

In prophylactic applications, compositions containing the invention adenoviruses, or cocktails thereof, are administered to a patient not presently in a neoplastic disease state to enhance the patient's resistance to recurrence of a cancer or to prolong remission time. Such an amount is defined to be a “prophylactically effective dose.” In this use, the precise amounts again depend upon the patient's state of health and general level of immunity.

Therapeutic Methods

Target Cancers: A key aspect of the instant invention is the discovery that the combination of adenovirus with certain chemotherapeutics causes a synergistic effect against cancer. Thus, therapy of neoplastic disease may be afforded by administering to a patient a composition consisting of adenovirus, wild type or a mutant, preferrably an E1b⁻ mutant, in combination with chemotherapy. The type of chemotherapeutics that will be combined with adenovirus to produce a synergistic effect will vary depending on the type of cancer to be treated, and are readily determined by a skilled practitioner of this art. For example, in the case of head and neck cancer, discussed more in the Example, the preferred chemotherapeutic regime is the use of two chemotherapeutics, cisplatin and 5-fluorouracil.

Various cancers may be treated with the invention adenoviral/chemotherapy combination. For example but not by way of limitation, a human patient having a bronchogenic carcinoma, nasopharyngeal carcinoma, laryngeal carcinoma, small cell and non-small cell lung carcinoma, lung adenocarcinoma, hepatocarcinoma, pancreatic carcinoma, bladder carcinoma, colon carcinoma, breast carcinoma, cervical carcinoma, ovarian carcinoma, or lymphocytic leukemias may be treated by administering an effective antineoplastic dosage of adenovirus. The cancers preferrably treated by the invention adenovirus/chemotherapy combination are squamous cell carcinoma solid cancers, more preferably such are cancers of the head and neck.

Squamous cell carcinoma of the head and neck afflicts an estimated 125,000 patients annually in developed countries in Europe, North America, and the Far East. In the U.S., the annual incidence is estimated at 45,000 cases with 15,000 associated deaths. Head and neck cancers have been reported to harbor p53 mutations in 45-70% of cases; both alcohol and tobacco use are associated with these mutations. Primary therapy for localized disease is surgery and adjuvant radiotherapy.

Cancer recurs in approximately one-third of patients following surgery. In the majority of cases, they recur in the region of the original primary neoplasm and lead to severe morbidity due to pain and to oropharyngeal and laryngeal obstruction and the resultant difficulties in swallowing and speech. Once the cancer has recurred and/or metastasized, the patient is considered incurable. Palliative surgery is difficult and disfiguring, and further radiation therapy is not generally beneficial for more than a few months. Several chemotherapeutic agents have been used in recurrent squamous cell carcinoma of the head and neck. Combination regimens have been shown to induce responses in 30-40% of patients, but the therapy can be toxic and there is no clear impact on survival. Once a patient's cancer is refractory to chemotherapy and/or radiation therapy, the median life-expectancy is 3 months and cancer response rates to second or third-line chemotherapeutic agents are ≦15%. Thus, the instant invention fulfills an urgent need for more effective therapies for these terminally ill patients.

Suspensions of infectious adenovirus particles may be applied to neoplastic tissue by various routes, including intravenous, intra-arterial, intratumoral, intraperitoneal, intramuscular, subdermal, and topical. A adenovirus suspension containing about 10³ to 10¹² or more virion particles per ml may be inhaled as a mist (e.g., for pulmonary delivery to treat bronchogenic carcinoma, small-cell lung carcinoma, non-small cell lung carcinoma, lung adenocarcinoma, or laryngeal cancer) or swabbed directly onto the cancer (e.g., bronchogenic carcinoma, nasopharyngeal carcinoma, laryngeal carcinoma, cervical carcinoma) or may be administered by infusion (e.g., into the peritoneal cavity for treating ovarian cancer, into the portal vein and/or hepatic artery for treating hepatocarcinoma or liver metastases from other non-hepatic primary cancers) or other suitable route, including direct injection into a cancer mass (e.g., a breast cancer), enema (e.g., colon cancer), or catheter (e.g., bladder cancer).

To obtain significant synergistic effect of adenovirus and chemotherapy, adenovirus is preferably administered over several consecutive days, more preferably it is administered daily over a three to seven day period. In the case of squamous cell cancer, for example cancer of the head and neck, the most preferred administration schedule is consecutively over five days. However, it is important to note that the number of actual days will vary depending on the type of cancer treated, and that a skilled practitioner of this art, knowing this from the disclosure set forth herein, could readily determine the best administration regimen to achieve maximum synergistic effect.

Adenoviral therapy using the instant invention adenoviruses may be combined with other antineoplastic protocols, such as gene therapy. As mentioned above, adenovirus constructs for use in the instant invention may exhibit specific cancer cell killing, preferably though the expression of pro-drug activator genes driven off a tissue specific promoter.

Also, in the event that the instant adenoviral vectors, including wild type or mutant viruses elicit an immune response that dampens their effect in a host animal, they can be administered with an appropriate immunosuppressive drug to maximum their effect.

Administration of Chemotherapy: Chemotherapy may be administered by methods well known to the skilled practitioner, including systemically, direct injection into the cancer, or by localization at the site of the cancer by associating the desired chemotherapeutic agent with an appropriate slow release material or intra-arterial perfusing the tumor.

The preferred chemotherapeutic agent is cisplatin, and the preferred dose may be chosen by the practitioner based on the nature of the cancer to be treated, and other factors routinely considered in administering cisplatin. Preferably, cisplatin will be administered intravenously at a dose of 50-120 mg/m² over 3-6 hours. More preferably it is administered intravenously at a dose of 80 mg/m² over 4 hours. Additionally, it is administered preferably on day 1 of treatment with adenovirus.

A second chemotherapeutic agent, which is preferably administered in combination with cisplatin is 5-fluorouracil. The preferred dose of 5-fluorouracil is 800-1200 mg/m² per day for 5 consecutive days (continuous infusion).

Synergistic Effect of Adenovirus/Chemotherapy: An aspect of the instant invention is that the anti-cancer effect observed for the combination adenovirus/chemotherapy is greater than the effect of either agent alone; that is the effect is greater than additive. Thus, the combination adenovirus/chemotherapy has a synergistic anti-cancer effect. Table 1 shows the response rate for recurrent head and neck cancers in five human clinical trials for patients treated with chemotherapy. The average response, consisting of complete responses (CR) and partial responses (PD) for the trials was 37%. A discussion of the trials is presented in Paredes, J., et al., Prospective randomized trial of high-dose cisplatin and fluorouracil infusion with or without sodium diethyldithiocaramate in recurrent and/or metastatic squamous cell cancer of the head and neck, Journal of Clinical Oncology. 6:955-962, 1988; Jacobs, C., et al., Phase III randomized study comparing cisplatin and fluorouracil as single agents and in combination for advanced squamous cell carcinoma of the head and neck, Journal of Clinical Oncology. 10:257-263, 1992; Forastiere, A., et al., Randomized comparison of cisplatin plus fluorouracil and carboplatin plus fluorouracil versus methotrexate in advanced squamous cell carcinoma of the head and neck: A southwest oncology group study, Journal of Clinical Oncology. 10:1245-1251, 1992; Schrijvers, D., et al., Phase III trial of modulation of cisplatin/fluorouracil chemotherapy by interferon alfa-2b in patients with recurrent or metastatic head and neck cancer, Journal of Clinical Oncology. 16:1054-1059, 1998; LHNOG A phase III randomized trial of cisplatin, methotrexate, cisplatin+methotrexate and cisplatin+5-FU in end stage squamous carcinoma of the head and neck, British Journal of Cancer. 61:311-315, 1990; Clavel, M., et al., Randomized comparison of cisplatin, methotrexate, bleomycin, and vincristine (CABO) versus cisplatin and 5-fluorouracil (CF) versus cisplatin (C), in recurrent or metastatic squamous cell carcinoma of the head and neck. A Phase III study of the EORTC head and neck cancer cooperative group, Annals of Oncology. 5:521-526, 1994.

TABLE 1
Phase III Studies of Recurrent Head and Neck Cancer

| | | PATIENTS | | |
|---|---|---|---|---|
| STUDY | YEAR | ENTERED | PR + CR | CR |
| LHNOG | 1990 | 39 | 31% | 0 |
| FORASTIERE | 1992 | 87 | 32% | 6% |
| JACOBS | 1992 | 63 | 40% | 8% |
| CLAVEL | 1994 | 108 | 34% | 0 |
| SCHRIJVERS | 1998 | 122 | 47% | 11% |
| TOTAL | | 419 | 37% | |

In comparison to chemotherapy, the response rate for recurrent head and neck cancer to adenovirus alone is 26%. Again this represents complete and partial responses. These results are shown in Table 2.

TABLE 2
ONYX-015 HEAD AND NECK CANCER STUDIES:
ALL SINGLE AGENT ONYX-015 DATA RECURRENT,
REFRACTORY HEAD AND NECK CANCER

| # of patients | CR | PR | % Response |
|---|---|---|---|
| 23 | 2 | 4 | 26 |

In contrast to the response rate to chemotherapy or adenovirus alone, the response rate to chemotherapy and adenovirus for recurrent head and neck cancer is about 90%, as described more in detail in the Example.

The Example which follows is illustrative of specific embodiments of the invention, and various uses thereof. It is set forth for explanatory purposes only, and is not to be taken as limiting the invention.

### EXAMPLE

### Treatment of Squamous Cell Carcinoma of the Head and Neck

Patients suffering from squamous cell carcinoma of the head and neck were treated with the adenovirus E1b⁻ mutant, d1 1520, also referred to herein as ONYX-015, and chemotherapy as described below. D11520 is described by Berk, in Virology 156: page 107 (1987) and can be obtained from Dr. Arnold Berk, University of California at Los Angeles, Calif.

Inclusion Criteria: Patients were enrolled in the clinical trials based on certain inclusion criteria. The cancer status had to be histologically confirmed squamous cell carcinoma of the head and neck, including the oral cavity, pharynx and larynx. It had to be recurrent disease in which the recurrent cancer has not been previously treated with chemotherapy. Recurrent disease refers to cancer which progresses following primary therapy with surgery and/or radiation (i.e. includes primary refractory cancers).

Patients who have received prior chemotherapy for their primary head and neck cancer(s) and who have not progressed within four weeks following the completion of this primary chemotherapy regimen were also eligible for inclusion.

Further, the entire cancer had to be amenable to direct injection with virus, and the cancer had to be amenable to measurement clinically and/or radiographically. Also, the cancer had to be considered uncurable by surgery (as defined by attending surgeon) or radiation therapy.

For patients with cancers that were measured radiographically and are not clearly evaluable, baseline CT scans were evaluated. If a cancer was clearly measurable on CT scan as judged by the Principal Investigator, the patient was enrolled. If the cancer was not measurable by CT scan, an MRI scan was performed at the site and subsequently evaluated. If the cancer was not measurable by CT scan, MRI scan or physical exam, the patient was not be enrolled.

Other inclusion criteria were a Karnofsky Performance Status of ≧70%, and a life expectancy of ≧3 months.

Administration of ONYX-015: ONYX-015 was formulated as a sterile viral solution in TRIS buffer (10 mM TRIS pH 7.4, 1 mM MgCl₂, 150 mM NaCl, 10% glycerol). The ONYX-015 solution contains no preservative. The virus may be stored frozen prior to use. The total dose of ONYX-015 administered was 10¹⁰ pfu daily for 5 days. On day 1, ONYX-0 15 treatment and chemotherapy initiation routinely occurred in the morning. ONYX-0 15 was administered before initiation of chemotherapy on day 1. Virus solution was thawed and initially diluted with a physiological solution to the appropriate titer. Thawed virus was maintained at 2° to 8° C. during dilution and handling, except for warming to room temperature immediately prior to administration. The virus solution, after dilution to the appropriate titer, was then further diluted to a final volume equivalent to 30% of the estimated cancer volume to be injected. Cancer volume was estimated by taking the product of the maximal cancer diameter, its perpendicular and the estimated depth, and dividing by two. This estimate was made with ultrasound, MRI, CT scan, and/or clinical examination. For cancers that had developed central ulceration, the estimated cancer volume was adjusted by subtracting the volume of the ulcerated area (latter estimated by taking the product of the maximal diameter of the ulcerated area, its perpendicular and estimated depth, and dividing by two). Dilutions were performed just prior to cancer injection.

The target cancer(s) were mapped into 5 equal-sized, equally-spaced sections through the use of a cancer template map. Prior to ONYX-015 injection, patients may be pre-medicated with local or systemic analgesics, at the Investigators discretion, based on the patient's pre-existing pain or on anticipated pain from the injection. On day one of each treatment cycle, aspiration of central necrotic tissue/fluid within the cancer was generally attempted prior to injection. At each of the five treatment sessions, injection (using a 25 gauge or smaller needle) was directed to one of the five cancer sections and was done in a manner to distribute equal volumes of virus throughout the entire cancer section. While injecting the virus, the syringe was withdrawn in order to distribute the injection volume equally along the entire needle track. Importantly, the injection technique used caused the virus to be distributed out to the spreading edge and to the deep component of the cancer. After injection, gentle pressure was applied to the injected areas for 2-3 minutes, if necessary, to prevent leakage of the virus solution out of the injection site.

Administration of Chemotherapeutics, Cisplatin and 5-FU:

Cisplatin 80 mg/m² was administered IV over 4 hours (±1 hour). As mentioned above, on day 1, Cisplatin treatment occurred after treatment with ONYX-015 on day 1.

5-FU 1,000 mg/m² per day was administered in up to 2 liters saline solution if given in a hospital, or in up to 0.5 liter saline solution if given by portable pump in a non-hospital environment. Administration was IV continuous infusion per day on days 1-5 (i.e. 5,000 mg/m² total dose/cycle).

Repeat Treatment:

Patients received repeat treatments with ONYX-015 at the same dosage and cisplatin, 5-FU up to a total of 5 cycles of treatment, administered every 3 weeks (counting from day 1 of the previous treatment cycle), if they showed no evidence of progressive disease at the target cancer site following at least 2 treatment cycles with ONYX-015.

Cancer Response Criteria: Using the following standard criteria, response was assessed separately on the injected target cancer. Duration of response and progression-free survival was determined. Classical/standard cross-sectional cancer measurements were used to assess response and were the following: [maximal cancer diameter×perpendicular diameter]. Ulcerated cancer areas were subtracted from the overall area. Computer-assisted cross-sectional measurements were performed by digital image analysis. Physical exam measurements of cancer size were used to determine cancer response if these measurements are felt to be more accurate than radiographic scanning in a given patient.

Cancer response to viral/chemotherapy treatment was graded as follows:

Complete response (CR): complete disappearance of cancer at the assessed site(s)

Partial response (PR): regression of the cancer(s) by 50% but less than 100%

Minor response (MR): regression of the cancer by less than 50%.

In the case of a PR, Computer-assisted calculations of cross-sectional area did not include necrotic areas of cancer. Finally, all responses to treatment must last for at least 4 weeks to be before they are classified appropriately.

Results

Table 3 presents the results of ten patients treated with ONYX-015, and cisplatin and 5-fluorouracil.

TABLE 3
SUMMARY OF RESPONSE TO ONYX-015 TREATMENT
HEAD AND NECK PHASE II COMBINATION WITH
CHEMOTHERAPY

| | | Weeks in | | |
|---|---|---|---|---|
| | Date of | study | Neoplasm | Local Neoplasm |
| Patient | Treatment | (cycles) | size (cm) | Response |
| 1. | 11/19/97 | 18 weeks | Left neck | *Complete Response |
| | 12/07/97 | (6 cycles) | 2.5 × 3.0 | |
| | 12/30/97 | | | |
| | 01/26/98 | | | |
| | 04/06/98 | | | |
| | 04/27/98 | | | |
| 2. | 02/02/98 | 13 weeks | 15 × 4 | Partial Response |
| | 02/23/98 | (5 cycles) | Submandibular | |
| | 03/16/98 | | | |
| | 04/13/98 | | | |
| | 05/03/98 | | | |
| 3. | 02/02/98 | 13 weeks | Submandibular | Partial Response |
| | 03/02/98 | (3 cycles) | 5.5 × 3.5 | |
| | 04/06/98 | | | |
| 4. | 02/09/98 | 12 weeks | Right neck | Partial Response |
| | 03/16/98 | (3 cycles) | 4.0 × 4.8 | |
| | 04/06/98 | | | |
| 5. | 02/16/98 | 11 weeks | Right neck | Complete Response |
| | 03/09/98 | (2 cycles) | 3.1 × 2.6 | |
| 6. | 03/09/98 | 9 weeks | Left neck | Partial Response |
| | 04/13/98 | (2 cycles) | 2.5 × 3.2 | |
| 7. | 03/16/98 | 8 weeks | Left tongue | Partial Response |
| | 04/06/98 | (2 cycles) | 2 × 2.5 | |
| 8. | 03/16/98 | 8 weeks | Left neck | Minor Response |
| | 04/13/98 | (3 cycles) | 5 × 4 | |
| | 05/03/98 | | | |
| 9. | 03/23/98 | 6 weeks | Forehead | Partial Response |
| | 04/20/98 | (2 cycles) | 3 × 3 | |
| 10. | 03/30/98 | 6 weeks | Right neck | Partial Response |
| | 04/20/98 | (2 cycles) | 3.9 × 3.9 | |

It is readily apparent from the data in Table 3 that 9 out of 10 patients responded to treatment. This 90% response rate indicates a synergistic effect against the target head and neck cancers based on historical response rates for cisplatin and 5-fluorouracil of 37% (Table 1), and for adenovirus alone of 26% (Table 2).

The invention now being fully described, it will be apparent to one of ordinary skill in the art that many changes and modifications can be made thereto without departing from the spirit or scope of the appended claims.

1. A composition of matter comprising adenovirus and at least one chemotherapeutic.

2. A composition of matter as described in claim 1 wherein said adenovirus is Onyx 015.

3. A composition of matter as described in claim 2 wherein said chemotherapeutic comprises cisplatin.

4. A composition of matter as described in claim 1 wherein said chemotherapeutic comprises cisplatin and 5-fluorouracil.

5. A method for treating cancer in a patient in need thereof comprising the steps of:
(a) contacting said cancer with adenovirus, and
at least one chemotherapeutic in amounts and for a time sufficient to substantially kill said cancer, and, if desired, repeating step (a) to prevent said cancer from reoccurring.

6. A method as described in claim 5 wherein said cancer is squamous cell cancer.

7. A method as described in claim 6 wherein said squamous cell cancer is of the head and neck.

8. A method as described in claim 7 wherein contacting said cancer with adenovirus comprises administering said adenovirus by direct injection into said cancer of said patient at a dose of about 10⁸-10¹² plaque forming units.

9. A method as described in claim 5 wherein contacting said cancer with adenovirus comprises administering said adenovirus intravenously into said patient.

10. A method as described in claim 5 wherein contacting said cancer comprises
administering to said patient said chemotherapeutic after said adenovirus.

11. A method as described in claim 5 wherein said chemotherapeutics comprise cisplatin and 5-fluorouracil.
